(19) United States
(12) Patent Application Publication
(10) Pub. No.: US 2011/0281808 A1
(43) Pub. Date: Nov. 17, 2011

(54) Composition and Method for Increasing Pre Workout Thermogenics

(75) Inventors: Michael Kichuk, Toronto (CA); Jason Bell, Toronto (CA); Stephen Brown, Toronto (CA); Shan Chaudhuri, Toronto (CA)

(73) Assignee: ALLMAX NUTRITION INC., Toronto (CA)

(21) Appl. No.: 12/780,217

(22) Filed: May 14, 2010

(51) Int. Cl. A61K 31/7048; A61P 3/00; A61P 25/00

(52) U.S. Cl. 514/27

(57) ABSTRACT

The present invention provides a composition for causing a simultaneous and synergistic increase in central nervous system activity and thermogenesis in a mammal, comprising Yerba Mate as a source of caffeine, hordenine and naringin. The present invention further provides a composition for causing a simultaneous and synergistic increase in central nervous system activity and thermogenesis in a mammal, comprising from 0.0001 g to 0.25 g of caffeine from Yerba Mate leaves, from 0.00001 g to 0.75 g of hordenine from barley and from 0.00001 g to 0.75 g of naringin sourced from grapefruit. The present invention finally provides a method for causing a simultaneous and synergistic increase in central nervous system activity and thermogenesis in an mammal prior to physical exercise, said method comprising providing a composition comprising a source of caffeine, hordenine and naringin and administering the composition to an mammal prior to physical exercise.

### FIELD OF THE INVENTION

The present invention relates to a nutritional supplement to prepare mind and body by stimulating the central nervous system and of a mammal prior to physical exercise, increase pre workout thermogenics and thereby improve the quality of exercise.

### BACKGROUND OF THE INVENTION

Human beings have been found to experience a few minutes of delay before steady-state oxygen uptake is established during exercise, illustrating that either feed-forward control of oxygen delivery is a weak influence or that there are considerable energy stores in skeletal muscle. Ventilation and circulation are kept at a low level at rest to allow graded increases to occur during exercise, as controlled by feedback from the muscles (‘the muscle pressor reflex’) as well as by a central nervous influence, termed ‘central command’. (Niels H. Secher. Central command and the onset of exercise. J Physiol 578.2 (2007) p 375-376).

Fatigue signals arising in the periphery during prolonged exercise may be modulated by the activity of serotoninergic neurons located within various areas of the brain, a hypothesis commonly referred to as the ‘central fatigue hypothesis’. An increase in the activity of serotoninergic neurons in specific parts of the brain may contribute to fatigue during prolonged exercise. Central fatigue could be delayed by nutritional manipulations. This central mechanism of fatigue could be related to the monoamine theory of thermoregulation. (Pitsiladis Y. P., et al—Hyperprolactinaemia during prolonged exercise in the heat: evidence for a centrally mediated component of fatigue in trained cyclists. Experimental Physiology (2002) 87.2, 215-226).

Caffeine affects responses to exercise. Caffeine elevates levels of blood lactate during intense exercise as well as increasing catecholamines. The increase in catecholamines in these circumstances enhances muscle glycogenolysis and this in turn generates greater anaerobic metabolism, resulting in greater lactate formation and muscle power output. Caffeine may increase lactate formation and possibly exercise performance during intense exercise. By contrast, when muscle power output is controlled, neither muscle nor blood lactate concentration nor net glycogenolysis is seen to be influenced by caffeine ingestion. Studies have demonstrated that caffeine ingestion can result in an increase in muscle endurance during intense exercise that leads to fatigue in about 5 min. (Jackman et al—Metabolic, catecholamine, and endurance responses to caffeine during intense exercise. J. Appl. Physiol. 81(4): 1658-1663, 1996).

High levels of spontaneous physical activity in lean individuals and the nonexercise activity thermogenesis (NEAT) derived from that activity appear to induce higher caloric expenditure than in obese individuals, while obesity is often characterized by dramatically reduced spontaneous physical activity and correspondingly lower caloric expenditure. Exercise, which has come to mean chosen physical activity, is universally cited as a means of weight control. The mechanics of exercise are primarily controlled by motor cortex, while the initiation of this form of physical activity is understood to be a higher cortical function. Shared neural systems may mediate both spontaneous physical activity and exercise, but spontaneous physical activity may be contrasted with exercise in that it may not be chosen and is not necessarily regulated by higher cortex. Need for motor control output still implies a brain site of regulation for spontaneous physical activity, but the initiation of that activity may originate in more autonomic brain sites such as the hypothalamus. (Kotz et al—Neuroregulation of nonexercise activity thermogenesis and obesity resistance. Am J Physiol Regul Integr Comp Physiol 294: R699-R710, 2008).

In the past, a pre-exercise high energy drink supplement (Redline Extreme containing caffeine anhydrous, beta-alanine, and vitamin C) has been tested on reaction time and anaerobic power in competitive strength/power athletes. In addition, the effect of the pre-exercise drink on subjective feelings of energy, fatigue, alertness and focus was also explored. Male strength/power athletes underwent two testing sessions administered in a randomized and double-blind fashion. A significant difference in reaction performance was seen between treatment and placebo groups. Subjective feelings of energy were significantly higher in the treatment group with an increase towards alertness. Results indicate a significant increase in reaction performance, with no effect on anaerobic power performance. In addition, ingestion of this supplement significantly improved subjective feelings of focus and energy in male strength/power athletes. (Hoffman et al—Examination of a pre-exercise, high energy supplement on exercise performance. Journal of the International Society of Sports Nutrition 2009, 6:2)

In one randomized, double-blind, placebo-controlled trial, otherwise healthy but overweight subjects were given a commercial weight loss product (Lean System 7) twice daily. Subjects taking the treatment had significant increase in resting metabolic rate (RMR). The product contained yerba mate, a source of caffeine. (John Zenk et al—Effect of Lean System 7 on metabolic rate and body composition. Nutrition 21 (2005): 179-185).

In situations preceding extended periods of repetitive, forceful muscular contractions, such as during exhaustive physical exercise, it would be advantageous for an individual to consume a pre workout nutritional supplement that will prepare mind and body of an individual by stimulating the central nervous system prior to work out and increase pre workout thermogenics and thereby improve the quality of exercise.

### SUMMARY OF THE INVENTION

The present invention thus provides a composition for causing a simultaneous and synergistic increase in central nervous system activity and thermogenesis in a mammal, comprising Yerba Mate as a source of caffeine, hordenine and naringin.

The present invention further provides a composition for causing a simultaneous and synergistic increase in central nervous system activity and thermogenesis in a mammal, comprising from 0.0001 g to 0.25 g of caffeine from Yerba Mate leaves, from 0.00001 g to 0.75 g of hordenine from barley and from 0.00001 g to 0.75 g of naringin sourced from grapefruit.

The present invention finally provides a method for causing a simultaneous and synergistic increase in central nervous system activity and thermogenesis in an mammal prior to physical exercise, said method comprising providing a composition comprising Yerba Mate as a source of caffeine, hordenine and naringin and administering the composition to an mammal prior to physical exercise.

### DETAILED DESCRIPTION OF THE INVENTION

The present invention is related to a nutritional supplement, comprising at least a therapeutically effective amount of source of caffeine, namely Yerba Mate leaf, N,N-dimethyltyramine (also called hordenine) and naringin. The ingredients of the present nutritional supplement act to prepare mind and body of an mammal by stimulating the central nervous system prior to physical exercise by increasing heart rate, increasing blood pressure and stimulating motor neurons of the spinal cord and by increasing pre workout thermogenics. This in turn improves the quality of exercise and helps individuals lose weight, in part, by stimulating norepinephrine release. Both composition and method are provided by the present disclosure.

Naringin is sourced from natural grapefruit juice that contains several flavonoid glycosides. Naringin (4′, 5, 7-tri-hydroxyflavonone 7-rhamno-glucoside) is the ‘bitter principal’ of grapefruit and is the most abundant of these compounds. Hordenine (C10H15NO) is a compound that is found in many grains, grassy plants, and in some species of hallucinogenic cactus. It is chemically related to the amino acid tyramine. Research on hordenine and fat loss is ongoing, but scientists hypothesize that hordenine helps people lose weight, in part, by stimulating norepinephrine release. Therefore, hordenine can be considered a central nervous system stimulant. Hordenine is thus often considered a central nervous system stimulant.

As used herein, the term ‘pre workout’ is understood to represent preparation of human body prior to intense exercise or workout. The main reason for including a warm-up at the beginning of a workout is to increase body temperature, raise the heart rate and prevent injury. Beginning a workout without warming and preparing the muscles will lead to pulled or strained muscles and dangerous changes in blood pressure. There are two types of warm-up: a general warm-up and a specific warm-up. A general warm-up is most common and involves performing an activity using movements that are not necessarily related to those used during the main exercise session. The most common example of a general warm-up can be found in fitness centers where an individual warms up on a stationary bike but then moves onto a treadmill for their main workout. A specific warm-up includes movements related to those used during the main workout. An example of a specific warm-up is a weight lifter performing squats, bicep curls and shoulder presses before lifting weights.

As used herein, the term ‘nutritional supplement’ includes dietary supplements, diet supplements, nutritional compositions, supplemental dietary and other compositions similarly envisioned and are not necessarily limited to conventional definitions of pharmaceutical interventions known in the art. Furthermore, ‘nutritional supplements’ as disclosed herein belong to a category of compositions having at least one physiological function when administered to a mammal by conventional and commonly known routes of administration.

### Yerba Mate

Yerba Mate is a known source of caffeine. It is a herb that has been consumed in South America for centuries. In studies conducted to determine the physiochemical characteristics for its standardizations, yerba mate was found to contain caffeine. In Argentina and Paraguay, the minimum value of caffeine found in Yerba Mate was 0.6%. (Scipioni et al—Physiochemical characterizations of different trademarks of compound Yerba Mate and their herbs. Brazilian archives of biology and technology. Vol 50, no. 4, pp 735-741, July 2007).

Yerba Mate has been used as a beverage since the time of the ancient Indians in Brazil and Paraguay. The active ingredients are xanthine alkaloids, specifically mateine—a chemical similar to caffeine, methylxanthine—specifically caffeine, theobromine, and theophylline. Like other xanthines, mateine is a mild central nervous system stimulant and a smooth muscle relaxant. The traditional use of yerba mate is to enhance athletic and cognitive performance and stimulate the nervous and muscular systems. (Leslie Taylor—Herbal secrets of the Rainforest, 2^{nd} edition, 2003).

The scientific name of yerba mate is Ilex paraguariensis, of the family Aquifoliaceae. Yerba mate grows in Brazil, Argentina, Paraguay and Uruguay. Besides caffeine, the leaves of yerba mate contain theobromine, theophylline, flavonoids, essential oils, tannins, steroids, triterpenes, resin and caffeic acid derivatives. It has historically been prepared into a mildly stimulant beverage called mate, by infusion of its dried leaves and stems.

In the 16^{th} century, it was reported that Guarani Indians of Paraguay brewed a Yerba Mate tea leaf that ‘produced exhilaration and relief from fatigue.’ It has a long history of use worldwide. In Europe, it is used for weight loss, treating physical and mental fatigue, treating nervous depression, pains and fatigue-related headaches. Yerba Mate is the subject of a German monograph which lists approved uses for mental and physical fatigue. In France, Yerba Mate is approved for the treatment of asthenia and as an aid in weight-loss programs. Yerba Mate is now cultivated in India, and the Indian Ayurvedic Pharmaceopia lists yerba mate for the treatment of psychogenic headaches, nervous depression, fatigue and rheumatic pains. In a Swiss study (1999) it was found that Yerba Mate was beneficial for human subjects as a weight-loss aid. Thermogenic effects have also been found in healthy individuals, where a drop in respiratory quotient was observed, indicating a rise in the proportion of fat oxidized. (Herbal Secrets of the Rainforest, 2^{nd} edition, 2003, Leslie Taylor).

Inclusion of Yerba Mate in a nutritional supplement increases caffeine levels in the body. This increased concentration of cellular caffeine will facilitate thermogenesis, thus improving warm up and increasing workout capacity. Caffeine increases both lipolysis, the breakdown of fat, and thermogenesis, calorie burning, for a period of time after ingestion. The increased thermogenesis leads to energy production required by the working muscle. The amount of caffeine regularly consumed by an individual increases the number of receptor sites that caffeine binds to in the central nervous system leading to more caffeine consumption being required over time to achieve the same stimulatory affect initially felt.

In an embodiment of the present invention, which is set forth in greater detail in the examples below, the nutritional composition comprises Yerba Mate.

A serving of the present nutritional composition thus preferably comprises from about 0.0001 g to about 0.25 g of Yerba Mate.

### Hordenine (N,N-dimethyl-4-hydroxyphenvlethylamine)

The flowers of Selenicereus grandiflorus cactus contain betacyanins and flavonolglycosides. About 8 glycosilated flavonoids have been identified in the stem of this cactus plant. Biogenic amines like tyramine, N-methyltyramine and N,N-dimethyltyramine (also known by the synonym hordenine) have also been found. This medicinal flower is known to increase heart rate, increase blood pressure and stimulate motor neurons of the spinal cord. A slight increase in heart rate, which helps in pre workout preparation, has been observed after oral administration of 500 mg hordenine to horses. Hordenine has a short half-life; only 24 minutes after ingestion has been determined in horses. The biogenic amines tyramine, N-methyltyramine and N,N-dimethyltyramine (i.e., hordenine) may be expected to occur in all plants that have the capability of metabolic proteolysis. Tyramine is a normal constituent of animal derived food and occurs in the organs of humans as a result of decomposition of the amino acid tyrosine. It is further methylated to N-methyltyramine and N,N-dimethyltyramine. Hordenine for example is contained in sprouting barley and may therefore be expected to be found in products thereof. (Selenicereus Grandiflorus—Summary Report of the European Agency for the Evaluation of Medicinal Products).

Hordenine (C10H15NO) is also found in many grains, grassy plants, and in some species of hallucinogenic cactus. It is chemically related to the amino acid tyramine. Research on hordenine and fat loss is ongoing, but scientists hypothesize that hordenine helps people lose weight, in part, by stimulating norepinephrine release. In this sense, hordenine is considered a central nervous system stimulant. (Bodybuilding.com)

Hordenine (N,N-dimethyl-4-hydroxyphenylethylamine) is a phenylethylamine alkaloid with antibacterial and antibiotic properties. It stimulates the release of norepinephrine in mammals. It is produced in nature by several varieties of plants in the family Cactaceae and by some in Acacia.

Hordenine has been known as the predominant alkaloid in roots of germinated barley. Hordenine is synthesized by the successive N-methylation of tyramine. The formation of tyramine methylpherase during barley germination has been studied. This enzyme is distributed uniformly along the entire length of the roots. The appearance and disappearance of the enzyme during the first month of the seedling growth corresponds with the levels of N-methyltyramine and hordenine during this period. Sufficient enzymatic activity is present to account for the synthesis of the entire alkaloid content of the roots. (Alkaloid and Plant Metabolism. Jay Mann et al. The Journal of Biological Chemistry. February 1963).

Hordenine has also been obtained from the marine algae Phyllophora crispa. In a GC-MS analysis of the algae, N-acetylphenylethylamine and hordenine were found and identified by comparison of the mass spectra with library data of mass spectra. (N-acetyltyramine from Phyllophora crispa. Aline Percot et al. Acta Pharmaceutica Sciencia. 51: 9-14. 2009)

The effect of hordenine on the central nervous system of cats under anaesthesia has been studied and it was determined that hordenine has similar qualitative and quantitative effects on the response of muscles to motor-nerve stimulation. It increases muscle tone and was seen to cause knee jerks and convulsions, mainly by causing stimulation of the spinal cord. (Schweitzer et al. Action of Hordenine compounds on the central nervous system. J. Physiol. (1938) 92, 422-438). Hordenine has thus been proven to prepare mind and body of an individual for exercise by stimulating the central nervous system, thus leading to a better and effective workout.

In an embodiment of the present invention, which is set forth in greater detail in the examples below, the nutritional composition comprises hordenine. A serving of the nutritional composition comprises from about 0.00001 g to about 0.75 g of hordenine.

### Naringin (4′, 5, 7-tri-hydroxyflavonone 7-rhamno-glucoside)

Natural grapefruit juice contains several flavonoid glycosides. Naringin (4′, 5, 7-tri-hydroxyflavonone 7-rhamno-glucoside) the so called ‘bitter principal’ of grapefruit, is the most abundant of these compounds. Naringin works by blocking key chemicals called enzymes that act like pairs of molecular scissors and chop up certain supplements, most notably caffeine. By blocking these scissors, naringin allows substances like caffeine to work for a longer period of time. (Bodybuilding.com)

Following oral administration to mammal, naringin is thought to be converted to the aglycone naringenin in the gut. A study was conducted to investigate the effect of grapefruit juice on CYP1A2 in mammal. Caffeine metabolism was used as a marker substrate for CYP1A2 activity both in vivo and in vitro. (Fuhr et al—Inhibitory effect of grapefruit juice and its bitter principal, naringenin, on CYP1A2 dependent metabolism of caffeine in man. B. J. clin PharmaC. (1993), 35, 431-436).

Naringin is metabolized to the flavanone naringenin in humans. Both naringenin and hesperetin, which are the aglycones of naringin and hesperidin, occur naturally in citrus fruits.

The flavanones from grapefruit—naringenin and hesperetin exhibit estrogenic, anticarcinogenic and antioxidative properties. Methods have been developed allowing for analysis of naringenin and hesperetin from plasma and urine. Flavanones were analyzed by HPLC and electrochemical detection. The result showed that naringenin is bioavailable from grapefruit juice. (Erlund et al—Plasma kinetics and urinary excretion of the flavanones naringenin and hesperetin in humans. J. Nutr. 131: 235-241, 2001).

The effects of grapefruit juice and naringenin on the activity of the human cytochrome P450 isoform CYP1A2 have been evaluated and it was found that vitro naringenin is a potent competitive inhibitor of caffeine 3-demethylation by human liver microsomes. In vivo grapefruit juice decreased the oral clearance of caffeine by 23% and prolonged its half-life by 31%. The competitive inhibition of caffeine 3-demethylation by naringenin indicates that the effects of grapefruit juice were, at least in part, caused by this flavanone. (Fuhr et al. Inhibitory effect of grapefruit juice and its bitter principal, naringenin, on CYP1A2 dependent metabolism of caffeine in man. Br. J. clin. Pharmac. (1993), 35, 431-416).

The present inventors have found that the oral administration of naringin from grapefruit acts synergistically with Yerba Mate (source of caffeine) to extend the half life of caffeine and extend thermogenesis and thus offer better workout when the nutritional composition is consumed prior to the workout.

In an embodiment of the present invention, which is set forth in greater detail in the examples below, the nutritional composition comprises naringin. A serving of the nutritional composition thus preferably comprises from about 0.00001 g to about 0.75 g of naringin.

In various embodiments of the present invention, which are set forth in greater detail in the examples below, the present nutritional supplement comprises yerba mate as a source of caffeine, naringin and hordenine. The present nutritional supplement is preferably provided in any acceptable and suitable oral dosage form as commonly known in the art. The nutritional supplement of the present invention may further preferably be administered in a dosage form having controlled release characteristics, e.g. time-release. Furthermore, the controlled release may be in forms such as an immediate release of active constituents, rapid release of constituents, delayed release of active constituents, gradual release of active constituents, or prolonged release of active constituents. Such active constituent release strategies extend the period of bioavailability or target a specific time window for optimal bioavailability.

According to various embodiments of the present invention, the nutritional supplement may be consumed in any form. For instance, the dosage form of the nutritional supplement may be provided as, e.g., a powder beverage mix, a liquid beverage, a ready-to-eat bar or ready-to-drink beverage, a capsule, a liquid capsule, a tablet, a caplet, or as a dietary gel. The most preferred dosage forms of the present invention are as a powder beverage mix or as a capsule.

Furthermore, the dosage form of the present nutritional supplement may be provided in accordance with customary processing techniques for herbal and nutritional supplements in any of the forms mentioned above. Additionally, the nutritional supplements set forth in the following examples may contain any appropriate number and type of excipients, as is well known in the art.

The method of the present invention further comprises at least the step of administering to an individual a therapeutically acceptable amount of the composition of the present invention. Preferably, the composition is administered to the individual 15 to 45 minutes prior to physical exercise and more preferably 30 minutes prior to physical exercise.

Although the following example illustrates certain embodiments of the practice of the present invention, the examples should not be construed as limiting the scope of the invention. Other embodiments will be apparent to one of skill in the art from consideration of the specifications and examples.

### EXAMPLES

### Example 1

A nutritional supplement comprising the following ingredients per serving is prepared for consumption as a powder beverage mix two times daily prior to workout:

from about 0.0001 g to about 0.25 g of Yerba Mate and from about 0.00001 g to about 0.75 g of hordenine.

### Example 2

A nutritional supplement comprising the following ingredients per serving is prepared for consumption as a powder beverage mix two times daily prior to workout:

from about 0.0001 g to about 0.25 g of Yerba Mate and from about 0.00001 g to about 0.75 g of naringin

### Example 3

A nutritional supplement comprising the following ingredients per serving is prepared for consumption as a capsule two times daily prior to workout:

from about 0.0001 g to about 0.25 g of Yerba Mate and from about 0.00001 g to about 0.75 g of hordenine.

### Example 4

A nutritional supplement comprising the following ingredients per serving is prepared for consumption as a capsule two times daily prior to workout:

from about 0.0001 g to about 0.25 g of Yerba Mate, from about 0.00001 g to about 0.75 g of hordenine and from about 0.00001 g to about 0.75 g of naringin.

### Example 5

A nutritional supplement comprising the following ingredients per serving is prepared for consumption as a powder beverage mix two times daily prior to workout:

from about 0.0001 g to about 0.25 g of Yerba Mate, from about 0.00001 g to about 0.75 g of hordenine and from about 0.00001 g to about 0.75 g of naringin Example 6

A nutritional supplement comprising the following ingredients per serving is prepared for consumption as a capsule two times daily prior to workout:

from about 0.0001 g to about 0.25 g of Yerba Mate, from about 0.00001 g to about 0.75 g of hordenine and from about 0.00001 g to about 0.75 g of naringin

In the foregoing specification, the invention has been described with a specific embodiment thereof; however, it will be evident that various modifications and changes may be made thereto without departing from the broader spirit and scope of the invention.

What is claimed:

1. A composition for causing a simultaneous and synergistic increase in central nervous system activity and thermogenesis in a mammal, comprising Yerba Mate as a source of caffeine, hordenine and naringin.

2. The composition of claim 1, wherein Yerba Mate is present in the composition in an amount of from 0.0001 g to 0.25 g.

3. The composition of claim 1, wherein hordenine is present in the composition in an amount of from 0.00001 g to 0.75 g.

4. The composition of claim 1, wherein naringin is present in the composition in an amount of from 0.00001 g to 0.75 g.

5. The composition of claim 2, wherein the source of caffeine in Yerba Mate is Yerba Mate leaves.

6. The composition of claim 3, wherein hordenine is sourced from barley.

7. The composition of claim 3, wherein hordenine is sourced from Selenicereus grandiflorus

8. The composition of claim 3, wherein hordenine is sourced from Phyllophora crispa

9. The composition of claim 4, wherein naringin is sourced from grapefruit.

10. A composition for causing a simultaneous and synergistic increase in central nervous system activity and thermogenesis in a mammal, comprising from 0.0001 g to 0.25 g of caffeine from Yerba Mate leaves, from 0.00001 g to 0.75 g of hordenine from barley and from 0.00001 g to 0.75 g of naringin sourced from grapefruit.

11. The composition of claim 1, wherein the composition is provided with a time release mechanism.

12. A method for causing a simultaneous and synergistic increase in central nervous system activity and thermogenesis in an mammal prior to physical exercise, said method comprising providing a composition comprising Yerba Mate as a source of caffeine, hordenine and naringin and administering the composition to an mammal prior to physical exercise.

13. The method of claim 12, wherein Yerba Mate is present in the composition in an amount of from 0.0001 g to 0.25 g.

14. The method of claim 12, wherein hordenine is present in the composition in an amount of from 0.00001 g to 0.75 g.

15. The method of claim 12, wherein naringin is present in the composition in an amount of from 0.00001 g to 0.75 g.

16. The method of claim 13, wherein the source of caffeine in Yerba Mate is Yerba Mate leaves.

17. The method of claim 14, wherein hordenine is sourced from barley.

18. The method of claim 14, wherein hordenine is sourced from Selenicereus grandiflorus

19. The method of claim 14, wherein hordenine is sourced from Phyllophora crispa

20. The method of claim 15, wherein naringin is sourced from grapefruit.

21. The method of claim 12, wherein the composition is administered to the mammal from 15 minutes to 45 minutes prior to physical exercise.

22. The method of claim 21, wherein the composition is administered to the mammal 30 minutes prior to physical exercise.

23. The method of claim 12, wherein the method is provided with a time release mechanism.
